SEQUENCE LISTING (36 sequences)

SEQ ID NO: 1 - DNA, 39 bp, mouse
  misc_feature (1)..(39): forward primer sequence specific for miR-21
aattgaattcggtaccttggcattaagccccagcaaacc

SEQ ID NO: 2 - DNA, 36 bp, mouse
  misc_feature (1)..(36): reverse primer specific for miR-21
aattgaattctccaagtctcacaagacataaggacc

SEQ ID NO: 3 - DNA, 21 bp, mouse
  misc_feature (1)..(21): forward PCR primer sequence
ctgggaggtgataagcgatga

SEQ ID NO: 4 - DNA, 19 bp, mouse
  misc_feature (1)..(19): reverse PCR primer sequence
cgctgtcaccgtggtaggt

SEQ ID NO: 5 - DNA, 18 bp, mouse
  misc_feature (1)..(18): forward PCR primer sequence
ccctgcccttgacctcaa

SEQ ID NO: 6 - DNA, 21 bp, mouse
  misc_feature (1)..(21): reverse PCR primer sequence
gcctcagtctcatggttttgg

SEQ ID NO: 7 - DNA, 19 bp, mouse
  misc_feature (1)..(19): forward PCR primer sequence
acaggagaagggacgccat

SEQ ID NO: 8 - DNA, 21 bp, mouse
  misc_feature (1)..(21): reverse PCR primer sequence
gaagccctacagacgagctca

SEQ ID NO: 9 - DNA, 18 bp, mouse
  misc_feature (1)..(18): forward PCR primer sequence
gcagctggattttggaaa

SEQ ID NO: 10 - DNA, 20 bp, mouse
  misc_feature (1)..(20): reverse PCR primer sequence
gtggctggctctcattcaca

SEQ ID NO: 11 - DNA, 17 bp, mouse
  misc_feature (1)..(17): forward PCR primer sequence
cgcaaggcccccactac

SEQ ID NO: 12 - DNA, 19 bp, mouse
  misc_feature (1)..(19): reverse PCR primer sequence
aaagtgggctacttcgatt

SEQ ID NO: 13 - DNA, 20 bp, mouse
  misc_feature (1)..(20): forward PCR primer sequence
ggttgccaagccttatcgga

SEQ ID NO: 14 - DNA, 20 bp, mouse
  misc_feature (1)..(20): reverse PCR primer sequence
acctgctccactgccttgct

SEQ ID NO: 15 - DNA, 19 bp, mouse
  misc_feature (1)..(19): forward PCR primer
gcagtggctgaaccaagga

SEQ ID NO: 16 - DNA, 19 bp, mouse
  misc_feature (1)..(19): reverse PCR primer sequence
agcagtgagcgctgaatcg

SEQ ID NO: 17 - DNA, 18 bp, mouse
  misc_feature (1)..(18): forward PCR primer sequence
cccaggcgcaatgtcaat

SEQ ID NO: 18 - DNA, 19 bp, mouse
  misc_feature (1)..(19): reverse PCR primer sequence
ccccaaccagccacatagc

SEQ ID NO: 19 - DNA, 18 bp, mouse
  misc_feature (1)..(18): forward PCR primer sequence
tgtggctggctgctgtca

SEQ ID NO: 20 - DNA, 23 bp, mouse
  misc_feature (1)..(23): reverse PCR primer sequence
tcagaggccacttggtaaaaaac

SEQ ID NO: 21 - DNA, 21 bp, mouse
  misc_feature (1)..(21): forward PCR primer sequence
cacaagatgctgggacagtga

SEQ ID NO: 22 - DNA, 20 bp, mouse
  misc_feature (1)..(20): reverse PCR primer sequence
tccttgatggtggtgcatga

SEQ ID NO: 23 - DNA, 21 bp, mouse
  misc_feature (1)..(21): forward PCR primer sequence
gaaggcatccagacccgaaac

SEQ ID NO: 24 - DNA, 20 bp, mouse
  misc_feature (1)..(20): reverse PCR primer sequence
acccatggcggtgaccatgc

SEQ ID NO: 25 - DNA, 24 bp, mouse
  misc_feature (1)..(24): forward PCR primer sequence
tcaagtggcatagatgtggaagaa

SEQ ID NO: 26 - DNA, 21 bp, mouse
  misc_feature (1)..(21): reverse PCR primer sequence
tggctctgcaggattttcatg

SEQ ID NO: 27 - DNA, 19 bp, mouse
  misc_feature (1)..(19): forward PCR primer sequence
cagcactgccaagcatggt

SEQ ID NO: 28 - DNA, 20 bp, mouse
  misc_feature (1)..(20): reverse PCR primer sequence
accgaaacgctgatccaaag

SEQ ID NO: 29 - DNA, 23 bp, mouse
  misc_feature (1)..(23): forward PCR primer sequence
gaagacccacactcgaattcact

SEQ ID NO: 30 - DNA, 21 bp, mouse
  misc_feature (1)..(21): reverse PCR primer sequence
ccccacaggtttcacatttgt

SEQ ID NO: 31 - DNA, 36 bp, mouse
  promoter (1)..(36): Full-length forward sequence.  It is a primer sequence.
aaaaaaacgcgtggttttgtggcaaggtctccgtgt

SEQ ID NO: 32 - DNA, 36 bp, mouse
  promoter (1)..(36): Full-length forward sequence.  It is a primer sequence.
aaaaaactcgagtgtcaggagggcgaggctagtgca

SEQ ID NO: 33 - DNA, 64 bp, mouse
  promoter (1)..(64): SB-mut1 forward sequence.  It is a primer sequence.
ttaaaccagttcttgaattcactagtggtgataaatgtgggacttctcagaagtcattcatttt

SEQ ID NO: 34 - DNA, 64 bp, mouse
  promoter (1)..(64): SB-mut1 reverse sequence.  It is a primer sequence.
aaaatgaatgacttctgagaagtcccacatttatcaccactagtgaattcaagaactggtttaa

SEQ ID NO: 35 - DNA, 64 bp, mouse
  promoter (1)..(64): SB-mut2 forward sequence.  It is a primer sequence.
ttaaaccagttcttacaggaactagtggtgataaatgtgggacttaggcctagtcattcatttt

SEQ ID NO: 36 - DNA, 64 bp, mouse
  promoter (1)..(64): SB-mut2 reverse sequence.  It is a primer sequence.
aaaatgaatgactaggcctaagtcccacatttatcaccactagttcctgtaagaactggtttaa

(19) United States
(12) Patent Application Publication
(10) Pub. No.: US 2013/0065972 A1
(43) Pub. Date: Mar. 14, 2013

(54) METHODS FOR SCREENING Th2 INFLAMMATORY DISEASES

(71) Applicants: Alexander L. Dent, Avon, IN (US); Deepali Sawant, Indianapolis, IN (US)

(72) Inventors: Alexander L. Dent, Avon, IN (US); Deepali Sawant, Indianapolis, IN (US)

(73) Assignee: Indiana University Research and Technology Corporation, Indianapolis, IN (US)

(21) Appl. No.: 13/607,515

(22) Filed: Sep. 7, 2012

Related U.S. Application Data
(60) Provisional application No. 61/583,823, filed on Jan. 6, 2012.
(60) Provisional application No. 61/531,638, filed on Sep. 7, 2011.

(51) Int. Cl. C12Q 1/68

(52) U.S. Cl. CPC C12Q 1/686; C12Q 1/6837; C12Q 1/6841
USPC 514/789; 435/6.12; 506/9; 435/6.11

(57) ABSTRACT

The present invention provides a method for screening for a Th2 inflammatory disease in a subject comprising the steps of: assaying a miR-21 expression level in a biological sample from the subject, and comparing the miR-21 expression level in the biological sample from the subject to the miR-21 expression level in a control, wherein an increase in the miR-21 expression level in the biological sample from the subject compared to the miR-21 expression level in the control indicates the presence of Th2 inflammatory disease in the subject.

### STATEMENT OF GOVERNMENT SUPPORT

This invention was made with government support under R21AI092212-01 awarded by the National Institutes of Health. The government has certain rights in the invention.

### BACKGROUND

MicroRNAs (“miRNAs”) are a class of small (about 19-25 nucleotides in length), endogenous, non-coding RNAs transcribed within the introns of other genes or encoded separately as uniquely regulated RNA gene products. They can post-transcriptionally regulate gene expression by binding to their target messenger RNAs (“mRNAs”) and inhibiting translation and/or reducing mRNA stability. mRNAs target specific mRNAs through Watson-Crick base pairing that predominately involves only bases 2-8 of the miRNA, known as the “seed.” A single miRNA may bind to a number of target transcripts, and a single transcript may contain multiple interaction motifs for a single miRNA or for different miRNAs.

The first miRNA was identified in the early 1990s, and there are now about 1000 distinct miRNAs that have been identified in humans. Increasing evidence has implicated miRNAs as having causative roles in a variety of lung diseases (e.g., asthma, chronic obstructive pulmonary disease, fibrosis and lung cancer), which has driven investigations into their potential as therapeutic targets. Of particular interest herein is miR-21, also known as hsa-mir-21 or miRNA21, which is a mammalian microRNA encoded by the MIR21 gene. MiR-21 is located in an intergenic region of human chromosome 17 and is known to target tumor suppressors, as well as play an important role in the development of heart disease.

BCL6 is a potent sequence-specific transcriptional repressor originally identified as an oncogene in non-Hodgkin's B cell lymphoma. Insights about a critical role for BCL6 in the T cell lineage first came from studies in Bcl6-deficient (Bcl6−/−) mice that develop spontaneous Th2-type inflammatory disease and also exhibit pronounced Th2 responses when challenged with an antigen [1-3]. The most common manifestation of the inflammatory disease in Bcl6−/− mice is severe myocarditis and pulmonary vasculitis, with up to 80% of mice developing disease as early as four weeks of age. Most Bcl6−/− mice die before 12 weeks of age, and these mice typically show severe inflammatory disease. The mechanism by which Bcl6 regulates Th2 cytokine expression is not well understood, but involves post-transcriptional regulation of the Th2 transcription factor Gata3 [4].

Recently, Bcl6 was identified as the lineage-defining transcription factor for follicular helper T cells (Tfh), a subset of T helper cells that provides help to B cells and promotes the germinal center reaction [5-7]. Several mechanisms have been proposed for how Bcl6 acts as a master regulator of the Tfh lineage. Bcl6 may promote Tfh cell differentiation by inhibiting Th1, Th2 and Th17 differentiation programs [6, 7]. However, the effect of Bcl6 on Th17 differentiation is complex and is believed to involve gene regulation in both T cells and APCs [8]. Additionally, Bcl6 may direct Tfh cell differentiation via repression of the Tfh antagonistic transcription factor Blimp-15. Bcl6 may also promote Tfh cell differentiation by repressing a large cohort of micro-RNAs that normally repress Tfh cell development [7].

Regulatory T cells (Tregs) are an immune-regulatory subset of CD4⁺ T cells essential for the maintenance of peripheral tolerance and immune homeostasis. The transcription factor FOXP3 specifies the Treg lineage and maintains its functional program [9-11]. Treg cells function as potent inhibitors of T cell proliferation and of T cell-mediated inflammation. A multitude of mechanisms contribute to the Treg-mediated suppression: release of inhibitory cytokines (TGFβ, IL-10, IL-35), metabolic disruption (which includes competition with effector T cells for IL-2 consumption), dendritic cell-mediated suppressive mechanisms and cytotoxic mechanisms (encompassing granzyme-dependent and perforin-mediated killing) [12-19]. Treg function is highly orchestrated such that specific transcription factors regulate the ability of Tregs to inhibit discrete types of T cell responses. Thus T-bet uniquely controls the ability of Tregs to suppress Th1 responses [20], IRF4 regulates the ability of Tregs to suppress Th2 responses 21 and Stat3 directs the ability of Tregs to suppress Th17 responses [22].

### SUMMARY

In one aspect, a method for screening for a Th2 inflammatory disease in a subject is provided. The method comprises the steps of: assaying a miR-21 expression level in a biological sample from the subject by at least one of quantitative PCR, a microarray, or in situ hybridization, and comparing the miR-21 expression level in the biological sample from the subject to the miR-21 expression level in a control, wherein an increase in the miR-21 expression level in the biological sample from the subject compared to the miR-21 expression level in the control indicates the presence of a Th2 inflammatory disease in the subject.

In another aspect, a method for screening for eosinophilic esophagitis in a subject is provided. The method comprises the steps of: assaying a miR-21 expression level in a biological sample from the subject by at least one of quantitative PCR, a microarray, or in situ hybridization, and comparing the miR-21 expression level in the biological sample from the subject to the miR-21 expression level in a control.

In a further aspect, a method for screening for asthma in a subject is provided. The method comprises the steps of: assaying a miR-21 expression level in a biological sample from the subject by at least one of quantitative PCR, a microarray, or in situ hybridization, and comparing the miR-21 expression level in the biological sample from the subject to the miR-21 expression level in a control.

In an additional aspect, a method of treating a subject having or suspected of having a Th2 inflammatory disease is provided. The method comprises the step of administering at least one anti-inflammatory agent to the subject if miR-21 expression level in a biological sample from the subject is increased at least 5-fold relative to miR-21 expression level in a control.

### BRIEF DESCRIPTION OF THE FIGURES

FIG. 1. (a) Analysis of Treg cell compartment in the peripheral lymphoid organs of Bcl6−/− (“KO”) mice (black bars) and their wild-type (WT) littermates (white bars). Lymph node cells were surface stained for CD4, CD25 and stained intracellularly for FoxP3 and Helios. The frequency of CD4⁺CD25⁺FOXP3⁺ Tregs and Helios-CD4⁺CD25⁺FoxP3⁺ (iTregs) was quantified by flow analysis. N=3 per group (p=0.30 (NS) for CD4⁺CD25⁺FoxP3⁺ T cells and p=0.67 (NS) for iTregs). NS=non-significant (p>0.05). (b) Suppression of T cell proliferative responses by CD4⁺CD25⁺FoxP3⁺ Tregs sorted from Bcl6−/− (black bars) and wild-type (white bars) FoxP3-gfp mice following co-culture with wild-type CD25⁻FoxP3⁻ (Tresps) cells (grey bar) activated with anti-CD3 plus mitomycin-C treated APCs for 72 hours at different ratios (Treg/Tresp ratios—1:2 and 1:4). Proliferation assessed as [³H] thymidine incorporation. Data plotted as percent proliferation is representative of at least 3 independent experiments (mean and s.e.m. of triplicate cultures). (c) Suppression of Th1 inflammation in vivo by Bcl6−/− Tregs in a T cell transfer model of colitis. Colitis was induced following adoptive transfer of IL10−/− CD4⁺ T cells (Tresps) i.p. into Rag1−/− mice (dark grey line). Bcl6−/− FoxP3-gfp Tregs (black line) or their wild-type counterparts (light grey line) were co-transferred i.p. along with IL10−/− Tresps to separate cohorts of Rag1−/− mice to assess reversal of disease. Data represents percent weight change relative to initial weight averaged for 3-4 mice per cohort and assessed over a 4-wk time period (*p<0.05 and **p<0.01 for cohorts receiving Tresps alone relative to cohorts receiving Tresps with wild-type or Bcl6−/− Tregs). (d) Severity of colitis assessed by changes in colon length 4 weeks post-transfer to Rag1−/− mice as described in (c) (p=0.502(NS) between wild-type and Bcl6−/− Treg cohorts). (f) Co-transfer of Bcl6−/− Tregs (black bar) suppresses colitis induced following adoptive transfer of IL10−/− Tresps (grey bar) as effectively as the wild-type Tregs (white bar) as determined by the IBD scores. Colon sections from the 3 cohorts of Rag1−/− mice were scored 4 weeks post-transfer in a blinded-fashion on a scale of 0-6 (p=0.205 (NS) between wild-type and Bcl6−/− Treg cohorts). (a-f) *p<0.05 and **p<0.01 (two-tailed Student's t-test) (error bars=s.e.m.).

FIG. 2. (a) The table represents the miRs differentially expressed between sorted Bcl6−/− (“KO”) and wild-type Tregs with statistical significance analyzed by expression microarrays. N=Tregs from 3 wild-type mice and two Bcl6−/− mice. (b) Quantitative PCR validation of the selected set of miRs in Bcl6−/− Tregs (black bars) relative to wild-type Tregs (white bars) following activation for 16 hours with anti-CD3 and anti-CD28, normalized using sno202, sno234 and U6 as controls. Data are average expression from at least 3 different mice per group. (c) Quantitative PCR validation of miRNA expression (miR-21, miR-22, miR-146b) in sorted bone marrow chimera-derived Bcl6−/− Tregs and Tconv cells (CD45.1⁻) represented with expression in their respective wild-type Treg or Tconv counterparts (CD45.1⁺) set to 1 (dotted grey line), following activation for 16 hours with anti-CD3 and anti-CD28, with expression normalized to U6. Data are average expression from 6 different mice per group. (b-c)*p<0.05, **p<0.01, (two-tailed Student's t-test) (error bars, s.e.m.).

FIG. 3. (a) Quantitative PCR analysis of expression of miRs—21, 22 and 146b following ectopic expression of Bcl6 in T cells, normalized using sn0202, sno234 and U6 as controls. Naïve T cells were transduced with control RV and Bcl6 RV and the sorted RV⁺ T cells were re-stimulated for 4-6 hours to assess its effect on miR expression. Data are average expression from at least 3 different experiments. (b) Luciferase activity in Jurkat T cells co-transfected with full-length or SB1 and SB2 mutated miR-21 promoter driven luciferase reporters and expression constructs for CXN and CXN-Bcl6. Cells were stimulated with PMA and lonomycin for 24 hours prior to harvest and luciferase measurement. Results averaged from 5 independent experiments, where the basal activity of each promoter construct is set to 100 relative units. (a-b)*p<0.05, **p<0.01 (two-tailed Student's t-test) (error bars=s.e.m.).

FIG. 4. (a) Quantitative PCR analysis of miR-21 and Gata3, IL4, IL13 and lfng following ectopic expression of miR-21 RV in non-polarized (Th0) T cells, relative to control RV transduced cells. Sorted RV⁺ cells were re-stimulated with anti-CD3 and anti-CD28 for 4-6 hours for gene expression analysis. MiR expression normalized to U6, and expression of other genes normalized to btub. Data are averaged from at least 3 independent experiments. (b) ELISA for cytokines assayed from supernatants of scrambled control, miR-21 mimic and antagomiR-21 treated naïve T cells. Cells were cultured with the oligos (1 μM) over a 5-day period, then re-stimulated with anti-CD3 and anti-CD28 overnight for cytokine measurements. Data representative of 3 independent experiments. (c) Quantitative PCR analysis of miR-21, Spry1 and Gata3 following 12-16 hours treatment of naïve T cells with scrambled control and miR-21 mimic (1 μM). MiR expression normalized to U6, and expression of other genes normalized to tubb5. Data are averaged from at least 3 independent experiments. (d) Quantitative PCR analysis of Spry1 and Il-12a in sorted Tregs from Bcl6−/− (black bars) and wild-type (white bars) FoxP3-gfp mice, with expression normalized to btub. N=3 mice per group. (e) Quantitative PCR validation of Spry1 and Il-12a in the total lung RNA of Ova-sensitized and intranasally challenged wild-type recipient mice (grey bars) or immunized i.p. with Bcl6−/− Tregs (black bars) or wild-type Tregs (white bars) normalized using tubb5 as control. N=8-10 mice per group. (a-c)*p<0.05, **p<0.01 (two-tailed Student's t-test) (error bars, s.e.m.).

FIG. 5. (a-b) Quantitative PCR analysis of the 3 miRs up-regulated in Bcl6−/− Tregs in the total lung RNA (a) and serum (b) of Ova-sensitized and intranasally challenged wildtype recipient mice (grey bars) or immunized i.p. with Bcl6−/− Tregs (black bars) or wild-type Tregs (white bars) normalized using U6 as control. N=8-10 mice per group.

FIG. 6. MiR-21 is strongly elevated in the human allergic disease eosinophilic esophagitis. Quantitative PCR (QPCR) analysis of miR-21 (A) and miR-22 (B) expression in esophageal biospsy RNA and miR-21 in serum (C) from non-EoE controls and eosinophilic esophagitis (EoE) patients, normalized using U6 as control. Relative miR expression is shown, with average dCT values from control patient samples set as 1, and fold-change calculated by the ddCT method. MiR-22 was not detected at significant levels in serum from either EoE or non-EoE patients. N=18-20 subjects per group. ***p<0.001 (two-tailed Student's t-test) (error bars, s.e.m.).

FIG. 7. Serum miR-21 is increased in asthma. Quantitative PCR (qPCR) analysis of miR-21 (A) and miR-22 (B) expression in serum samples prepared from 5 year old patients presenting at 1 year of age with dermatitis and then monitored for allergy symptoms and the development of asthma. Relative miR expression was calculated as described in FIG. 6. (C) Pearson correlation analysis between IgE levels (in IU) and miR21 levels (fold-change from average non-asthmatic control). N=16 total patients, 8 with asthma and 8 without asthma. *p<0.05 (two-tailed Student's t-test) (error bars, s.e.m.).

FIG. 8. (a) ELISA for cytokines assayed from supernatants of miR-21 transduced Th2 cells (IL-4—20 ng/ml plus anti-IFNg-10 μg/ml), relative to control RV transduced cells. Cells were re-stimulated with anti-CD3 and anti-CD28 for 24 hours following sorting of RV⁺ Th2 cells for cytokine measurements. *p<0.05, (two-tailed Student's t-test) (error bars=s.e.m.). (b) Quantitative PCR analysis of miR-21 gene targets (Spry1, Il12a, Smad7, Pten, Pdcd4, Btg2) following 12-16 hours treatment of naïve T cells with scrambled control, miR-21 mimic and antagomiR-21 oligos (1 μM), normalized to tubb5 as control. (c) Quantitative PCR validation of Pdcd4 and Pten in the total lung RNA of Ova-sensitized and intranasally challenged wild-type recipient mice (grey bars) or immunized i.p. with Bcl6−/− Tregs (black bars) or wild-type Tregs (white bars) normalized using tubb5 as control (N=8-10 mice per group).

FIG. 9. Expression of scrambled control probe and miR-21 probe in the paraffin-embedded lung sections of Ova-sensitized and intranasally challenged wild-type recipient mice immunized i.p. with Bcl6−/− Tregs in 2 representative sections as determined by LNA-based in situ hybridization.

FIG. 10 includes Table 1.

FIG. 11 includes Table 2.

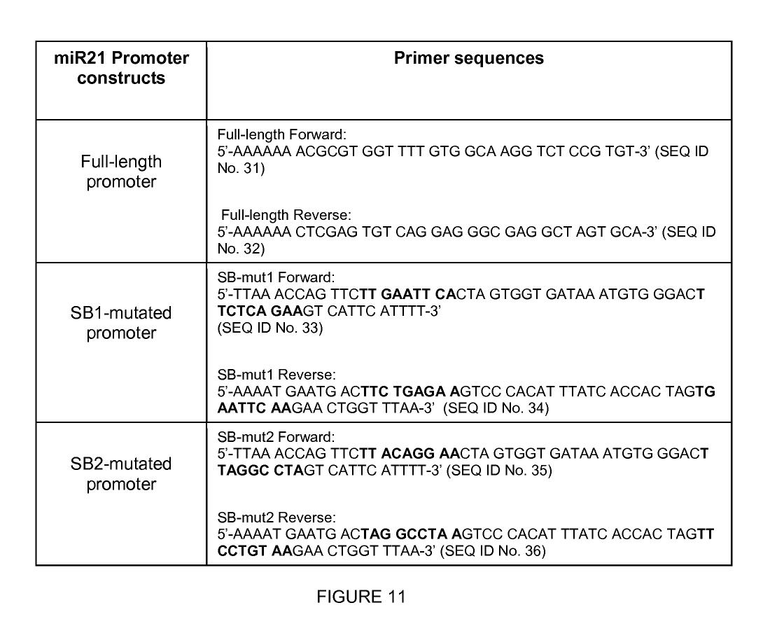

FIG. 12 includes Table 3.

### DEFINITIONS

When introducing elements of aspects of the invention or the embodiments thereof, the articles “a,” “an,” “the,” and “said” are intended to mean that there are one or more of the elements. The terms “comprising,” “including,” and “having” are intended to be inclusive and mean that there may be additional elements other than the listed elements. The term “or” means any one member of a particular list and also includes any combination of members of that list, unless otherwise specified.

As used herein interchangeably, a “miR-21” “microRNA-21,” or “miRNA-21” refers to the processed RNA transcript from a miR-21 gene. As the miR-21 gene products are not translated into protein, the term “miR-21 gene products” does not include proteins. The unprocessed miR-21 gene transcript is also called a “miR-21 precursor.” The miR-21 precursor can be processed by digestion With an RNAse (for example, Dicer, Argonaut, RNAse III (e.g., E. coli RNAse III)) into an active miR-21 molecule. This active RNA molecule is also called the “processed” miR-21 gene transcript or “mature” miR-21. The sequences of precursor and mature miR-21 are publicly available from the miRNA Registry (miRBase). The miRBase is described in Griffiths-Jones S., NAR, 2004, 32, D109-D111 and Griffiths-Jones S. et al. NAR, 2006, 23, D140-D144.

### DETAILED DESCRIPTION

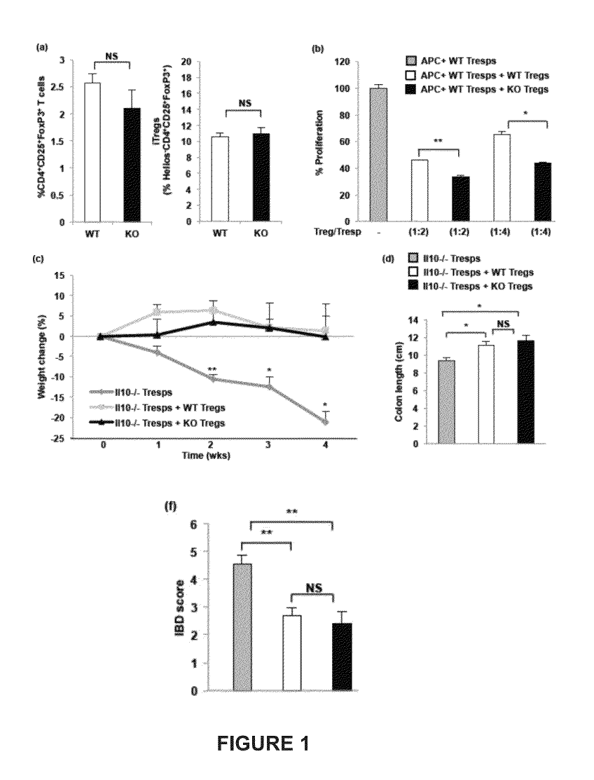

The present invention stems from the discovery that Bcl6 appears to be required to suppress Th2 genes in Tregs, in part by repressing the transcriptional activity of Gata3 (FIG. 1). Moreover, it has been discovered that Bcl6 represses miR-21 in Tregs, and that ectopic expression of miR-21 can promote Th2 expression; miR-21 has thus been identified as having altered expression in Th2 inflammatory diseases. Without being bound to any particular theory, it is believed that miR-21 regulates Th2 differentiation in a T cell autonomous manner. Importantly, miR-21 was found to be up-regulated in human allergic disease. For example, in eosinophilic esophagitis (EoE), miR-21 was elevated an average 50-fold over control biopsy levels. In contrast to miR-21, miR-22 and miR-146b were not elevated, indicating that miR-21 is a common biomarker for severe Th2-type inflammation. Moreover, miR-21 levels were increased almost 30-fold in the serum of EoE patients, indicating that elevated circulating miR-21 could serve as a non-invasive biomarker for Th2-type inflammation.

The above-mentioned association of miR-21 with particular diagnostic, prognostic and therapeutic features can be put to use in a method of screening for Th2 inflammatory diseases in a subject, such as asthma, allergic rhinitis, atopic dermatitis, food allergies, and eosinophilic esophagitis. The method is based on measuring the expression of miR-21, wherein an increase in expression may be indicative of a Th2 inflammatory disease.

The method includes the steps of assaying the miR-21 expression level in a biological sample from the subject to be screened for a Th2 inflammatory disease, comparing the assayed the miR-21 expression level to the miR-21 expression level in a control, for example a normal sample providing a control relative to the biological sample of the subject, and computing the differential expression of the miR-21 from the subject. The method can thus provide diagnostic evidence as to whether a subject has, or is at risk for developing, a Th2 inflammatory disease. As used herein, a “subject” can be any mammal that has, or is suspected of having, a Th2 inflammatory disease. In an exemplary set of embodiments, the subject is a human who has, or is suspected of having, a Th2 inflammatory disease.

A method of diagnosing a Th2 inflammatory disease in a subject may comprise, for example, measuring the level of miR-21 gene expression in a biological sample obtained from the subject. For example, a biological sample can be removed from a subject suspected of having a Th-2 inflammatory disease by conventional biopsy techniques. The biological sample may include, for example, blood, serum, a biopsy sample, a tissue sample, a cell suspension, saliva, oral fluid, cerebrospinal fluid, lymph, urine, gastric fluid, synovial fluid, mucus, sputum, and the like. A corresponding control sample, or a control reference sample, can be obtained from unaffected tissues of the subject, from a normal human individual or population of normal individuals, or from cultured cells corresponding to the majority of cells in the subject's sample.

The control sample is then processed along with the biological sample from the subject, so that the levels of miR-21 gene product produced in cells from the subject's sample can be compared to the corresponding miR-21 gene product levels from cells of the control sample. Alternatively, a reference sample can be obtained and processed separately (e.g., at a different time) from the biological sample and the level of a miR-21 gene product produced from a given miR-21 gene in cells from the biological sample can be compared to the corresponding miR-21 gene product level from the reference sample.

In a set of representative embodiments, the level of the miR-21 gene product in the biological sample is greater than the level of the corresponding miR-21 gene product in the control sample (i.e., the expression level of miR-21 in the biological sample is “upregulated”). As used herein, the expression level of miR-21 gene product is “upregulated” when the amount of miR-21 gene product in a biological sample from a subject is greater than the amount of the same gene product in a control sample. In another set of representative embodiments, the expression level of the miR-21 gene product in the biological sample is less than the level of the corresponding miR-21 gene product in the control sample (i.e., the expression of miR-21 in the biological sample is “downregulated”). As used herein, the expression level of a miR-21 gene is “downregulated” when the amount of miR-21 gene product produced from that gene in a biological sample from a subject is less than the amount produced from the same gene in a control biological sample.

The relative miR-21 gene expression level in the control and reference samples can be determined with respect to one or more RNA expression standards. The standards can comprise, for example, a Zero miR-21 gene expression level, the miR-21 gene expression level in a standard cell line, the miR-21 gene expression level in unaffected tissues of the subject, or the average level of miR-21 gene expression previously obtained for a population of normal human controls. An increase in the level of miR-21 gene expression in the biological sample obtained from the subject, relative to the level of a corresponding miR-21 gene product in a control, can be indicative of the presence of a Th2 inflammatory disease. In some embodiments, an increase in miR-21 expression level of at least 1.5-fold, 2-fold, 5-fold, 10-fold, 20-fold, 30-fold, 40-fold, 50-fold, 60-fold, 80-fold, or 100-fold relative to the control may be indicative of the presence of a Th-2 inflammatory disease.

The level of a miR-21 gene product in a biological sample can be measured using any technique that is suitable for detecting RNA expression levels in a sample. Suitable techniques (e.g., quantitative PCR (qPCR), microarrays, in situ hybridization, or older techniques such as Northern blot analysis) for determining RNA expression levels in a biological sample are well known to those of skill in the art. Among such techniques, the polymerase chain reaction (PCR) or more specifically qPCR (Q-PCR, qrt-PCR), is now commonly used to identify small quantities of a target nucleic acid in a sample. Development of PCR technologies based on reverse transcription and fluorophores permits measurement of DNA amplification during PCR in real time, i.e., the amplified product is measured at each PCR cycle. The data thus generated can be analyzed by computer software to calculate relative gene expression in several samples, or mRNA copy number.

In one set of representative embodiments, accordingly, the level of a miR-21 gene product is detected using quantitative PCR (qPCR). For quantitative PCR analysis, samples are usually first homogenized and the total RNA isolated. The RNA is then subject to reverse transcription for the synthesis of cDNA for subsequent analysis, and a PCR reaction with primers specific for miR-21 is started in a real-time PCR machine. The amplified DNA is detected as the reaction progresses in real time. Two common methods for detection of products in qPCR are: (1) non-specific fluorescent dyes that intercalate with any double-stranded DNA, and (2) sequence-specific DNA probes consisting of oligonucleotides that are labeled with a fluorescent reporter which permits detection only after hybridization of the probe with its complementary DNA target, such as the TaqMan hydrolysis probes (Applied Biosystems). Fluorescent reporter probes detect only the DNA containing the probe sequence; therefore, use of the reporter probe significantly increases specificity, and enables quantification even in the presence of non-specific DNA amplification.

qPCR may be used to quantify nucleic acids by two methods: relative quantification and absolute quantification. Relative quantification is based on internal reference genes to determine fold-differences in expression of the target gene. Absolute quantification gives the exact number of target DNA molecules by comparison with DNA standards.

The general principle of DNA quantification by real-time PCR relies on plotting fluorescence against the number of cycles on a logarithmic scale. A threshold for detection of DNA-based fluorescence is set slightly above background. The number of cycles at which the fluorescence exceeds the threshold is called the cycle threshold, C_{T}. During the exponential amplification phase, the sequence of the DNA target doubles every cycle. For example, a DNA sample whose C_{T} precedes that of another sample by 3 cycles contained 2³=8 times more template. However, the efficiency of amplification is often variable among primers and templates. Therefore, the efficiency of a primer-template combination is assessed in a titration experiment with serial dilutions of DNA template to create a standard curve of the change in C_{T} with each dilution. The slope of the linear regression is then used to determine the efficiency of amplification, which is 100% if a dilution of 1:2 results in a C_{T} difference of 1.

To quantify gene expression, the C_{T} for miR-21 is divided by C_{T} of RNA from a housekeeping gene in the same sample to normalize for variation in the amount and quality of RNA between different samples. This normalization procedure is commonly called the ΔΔC_{T}-method (“delta-delta CT, ddCT”) and permits comparison of expression of a gene of interest among different samples. When quantifying the expression of microRNAs such as miR-21, the data may be normalized to the expression of small nuclear RNAs (snRNA) or small nucleolar RNAs (snoRNA), such as sno202 and sno234.

In addition to qPCR, determining the levels of miR-21 gene products can be accomplished using the technique of in situ hybridization. This technique involves depositing whole cells onto a microscope cover slip and probing the nucleic acid content of the cell with a solution containing radioactive or otherwise labeled nucleic acid (e.g., cDNA or RNA) probes. This technique is particularly well-suited for analyzing tissue biopsy samples from subjects. Suitable probes for in situ hybridization of a given miR-21 gene product can be include, but are not limited to, probes having at least about 70%, 75%, 80%, 85%, 90%, 95%, 98% or 99% complementarity to a miR-21 gene product of interest, as well as probes that have complete complementarity to a miR-21 gene product of interest.

The levels of miR-21 gene transcripts can be quantified in comparison with an internal standard, for example, the level of mRNA from a “housekeeping” gene present in the same sample. A suitable “housekeeping” gene for use as an internal standard includes, but is not limited to, e.g., myosin or glyceraldehyde-3-phosphate dehydrogenase (G3PDH).

Methods of diagnosing a Th2 inflammatory disease as described herein may be useful in the diagnosis or prognosis of a Th2 inflammatory disease in a subject; predicting the susceptibility, onset or likely severity of a Th2 inflammatory disease in a subject; or in predicting the responsiveness of a subject to therapy. For example, a change in expression of miR-21 described herein relative to controls may be indicative that the subject is susceptible or has a high risk of suffering from a Th2 inflammatory disease relative to control members of the population.

In a set of embodiments, an increase in expression of miR-21 as set out herein may be indicative of allergic asthma. A method of diagnosing allergic asthma in a subject may comprise the step of assaying miR-21 expression in a sample of lung cells obtained from the subject. An increase in expression of miR-21 in the sample cells relative to controls is indicative that the subject suffers from allergic asthma. In another set of embodiments, an increase in expression of miR-21 may be indicative of EoE. A method of diagnosing EoE in a subject may comprise the step of assaying miR-21 expression in a sample of esophageal cells obtained from the subject. An increase in expression of miR-21 in the sample cells relative to controls is indicative that the subject suffers from EoE.

In a further set of embodiments, an increase in expression of miR-21 may be indicative of allergic rhinitis. A method of diagnosing allergic rhinitis in a subject may comprise the step of assaying miR-21 expression in a sample of nasal epithelium cells obtained from the subject. An increase in expression of miR-21 in the sample cells relative to controls is indicative that the subject suffers from allergic rhinitis. In another set of embodiments, an increase in expression of miR-21 may be indicative of atopic dermatitis. A method of diagnosing atopic dermatitis in a subject may comprise the step of assaying miR-21 expression in a sample of skin cells obtained from the subject. An increase in expression of miR-21 in the sample cells relative to controls is indicative that the subject suffers from atopic dermatitis. In an additional set of embodiments, an increase in miR-21 levels in the serum may be indicative of the presence of a Th2-type inflammatory disease in the subject, regardless of whether the disease is allergic asthma, EoE, allergic rhinitis, atopic dermatitis or another Th2-type inflammatory disease.

In other representative embodiments, a treatment plan for a Th2-type inflammatory disease can be developed if an increase in miR-21 expression level is detected. For instance, a treatment plan may be developed if the miR-21 expression level in a biological sample from the subject is found to be increased at least 5-fold relative to the mirR-21 expression level in a control. In other instances, the treatment plan may be developed if the increase in miR-21 expression level is one of at least 1.5-fold, 2-fold, 10-fold, 20-fold, 30-fold, 40-fold, 50-fold, 60-fold, 80-fold, or 100-fold relative to the control. In addition, an anti-inflammatory agent can be administered to a subject having or suspected of having a Th2-type inflammatory disease if the miR-21 expression level in a biological sample from the subject is found to be increased at least 5-fold relative to the mirR-21 expression level in a control. In other cases, an anti-inflammatory agent may be administered to the subject if the increase in miR-21 expression level is one of at least 1.5-fold, 2-fold, 10-fold, 20-fold, 30-fold, 40-fold, 50-fold, 60-fold, 80-fold, or 100-fold relative to the control.

### EXPERIMENTAL

Bcl6 Represses Micro-RNAs in Tregs

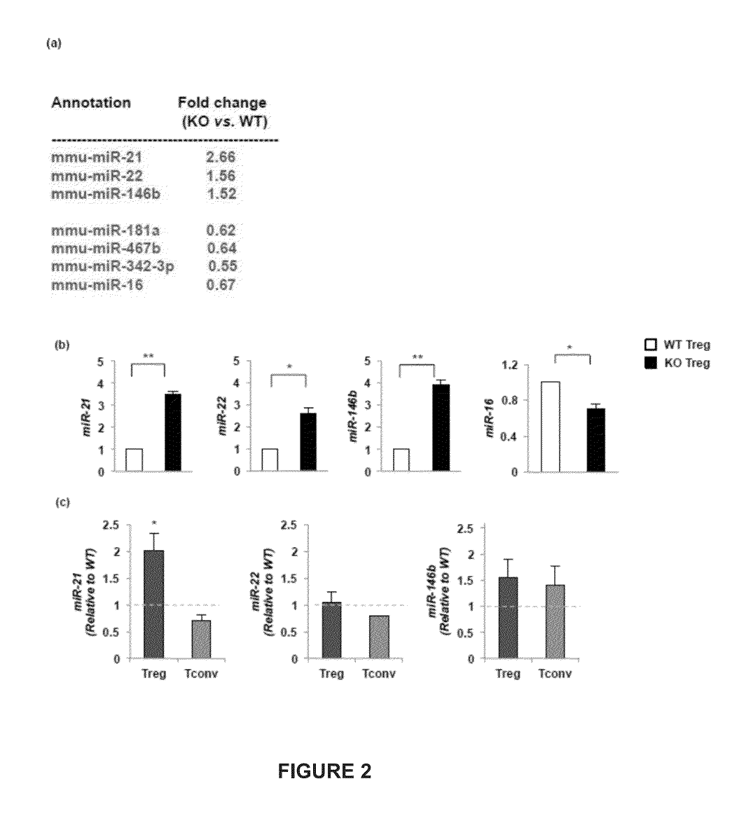

In order to establish whether Bcl6 regulated Treg lineage stability by repressing micro-RNAs in Tregs, a micro-RNA microarray approach was utilized, using RNA prepared from highly purified CD4⁺CD25⁺FoxP3⁺ Tregs from wild-type and Bcl6−/− mice, activated in vitro. As shown in FIGS. 2a and 2b, three micro-RNAs were up-regulated significantly in Bcl6−/− Tregs, and only miR-21 was up-regulated more than 2-fold in the Bcl6−/− Treg microRNA microarray. Two other micro-RNAs had increased expression in Bcl6−/− Tregs, miR-22 and miR-146b.

Next, in order to address if the increased micro-RNAs in Bcl6−/− Tregs were due to intrinsic regulation by Bcl6 in the Treg cells, Tregs from the chimeric mice described in FIG. 1 were tested for expression of these three miRs. As shown in FIG. 2c, only miR-21 was increased in Bcl6−/− Tregs from the chimera compared to wild-type Tregs in the same chimera, indicating that miR-21 is an intrinsic target of Bcl6 in Tregs. Thus, miR-22 and miR-146b do not appear likely to be directly regulated by Bcl6 in Tregs. Expression of miR21 was only increased in Bcl6−/− Tregs and not in Bcl6−/− wild-type conventional (Tconv) cells (FIG. 2c). Thus, Bcl6 appears to specifically repress miR21 in the Treg lineage.

Mir-21 is a Direct Target of Bcl6

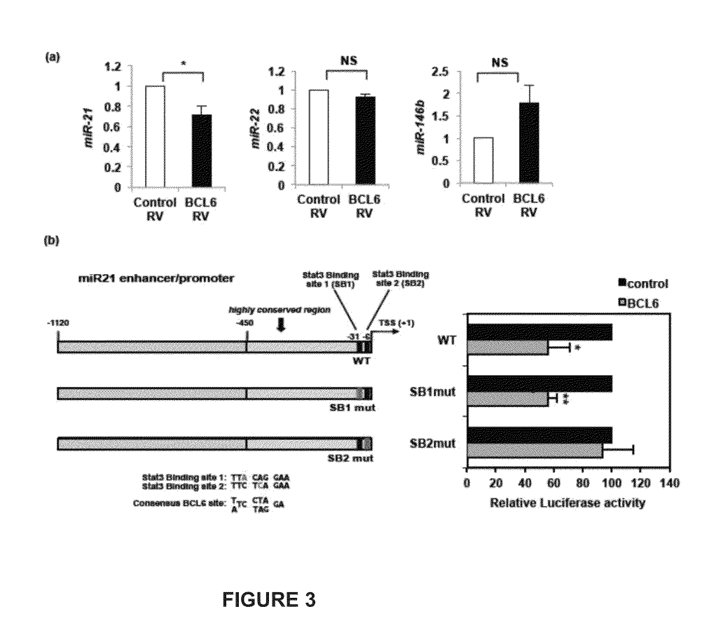

To further test the regulation of miR-21 by Bcl6, activated wild-type CD4⁺ T cells, which have low levels of Bcl6, were infected with a Bcl6-expressing retrovirus and tested for repression of miR-21, miR-22 and miR-146b. As shown in FIG. 3a, Bcl6 repressed miR-21 but not miR-22 and miR-146b, supporting the idea that miR-21, but not miR-146b or miR-22, is a direct target of Bcl6. These data are in agreement with the previous report on microRNAs repressed by Bcl6 in T cells [7].

Next, the miR-21 promoter sequence was analyzed, and two potential Bcl6 binding sites were found near the start site, which contains previously characterized Stat3 binding sites (FIG. 3b) [39, 40], and Stat3 positively regulates miR-21 in T cells [41]. It was found that Bcl6 could repress the wild-type miR-21 promoter significantly in a transient transfection assay in Jurkat cells (FIG. 3b). Mutation of the 5′ Stat3-binding site (SB1), had no effect on repression by Bcl6, whereas mutation of the 3′ Stat3-binding site (SB2), completely abolished repression by Bcl6. By comparing the sequence of the two Stat3 binding sites, it was found that the SB2 site has better conservation of the core Bcl6 recognition motif than SB1, thus explaining the differential effect of Bcl6 in regulating miR-21 via the two sites. These data suggest that Bcl6 and Stat3 may compete for binding to the miR-21 promoter, and that each oppose regulation by the other factor.

Mir-21 Can Promote Th2 Response

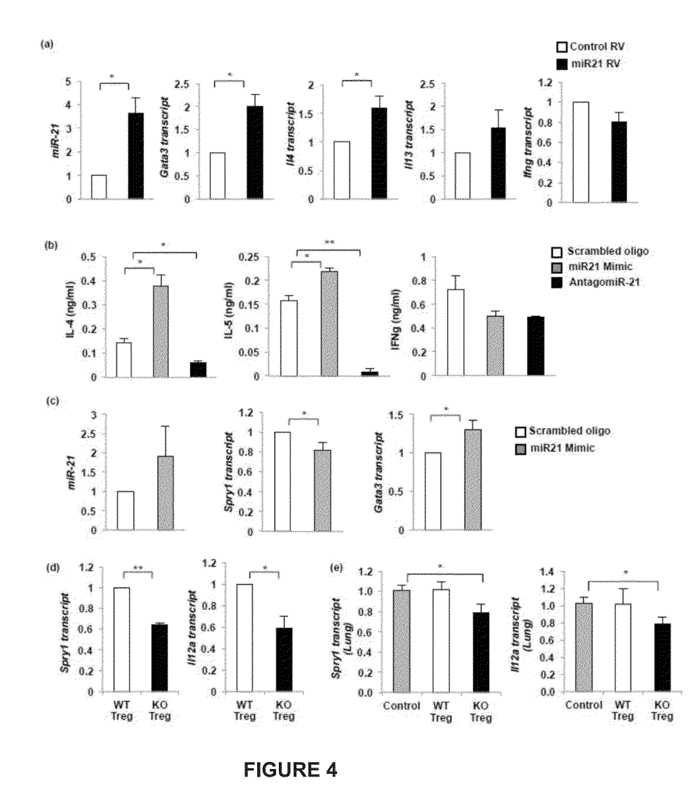

Although miR-21 has been linked to Th2 inflammation, it was reported as primarily elevated in cells of the myeloid lineage [38]. Thus, whether miR-21 regulates Th2 differentiation in a T cell autonomous manner has not been ascertained. To test this idea, a miR-21-expressing retrovirus was constructed, and used it to infect “naïve” CD4⁺CD62L⁺ T cells from wild-type mice, with the hypothesis that over-expression of miR-21 in T cells by retrovirus could recapitulate the elevated levels of miR-21 observed in Bcl6−/− Tregs. As shown in FIG. 4a, the retrovirus promoted a greater than 3-fold increase in miR-21 when expressed in T cells, which was comparable to the increased level observed in Bcl6−/− Tregs.

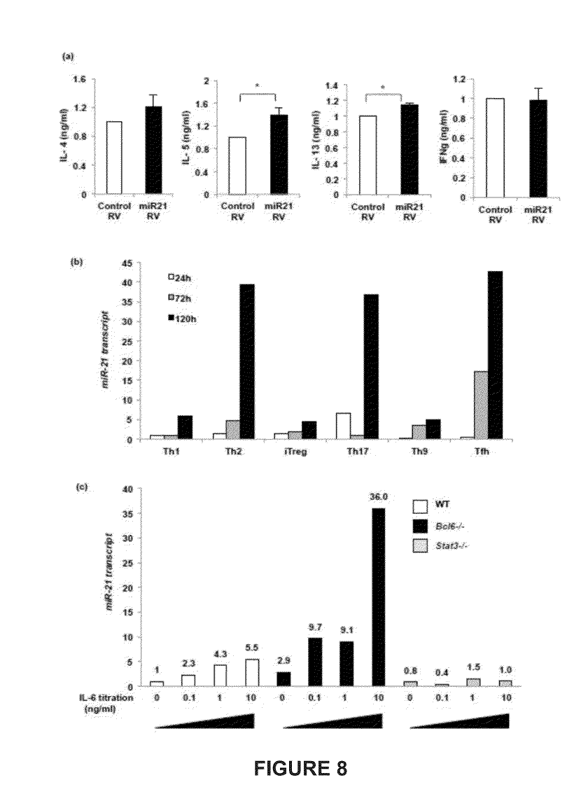

Next, a test was conducted to establish whether miR-21 could induce Th2 differentiation in cells cultured under “Th0” conditions, where no cytokines were added. As shown in FIG. 4a, miR-21 promoted increases in Gata3, IL4 and IL13, whereas expression of the Th1 cytokine Interferon-γ (Ifng) was mildly repressed by miR-21. It was also found that miR-21 could augment Th2 but not Th1 cytokine expression when overexpressed in Th2 cells (FIG. 8a). To further assess miR-21 activity in promoting Th2 differentiation, a synthetic miR-21 “mimic” was tested as well as an antisense miR-21 inhibitor “antagomiR-21” in Th0 differentiation cultures. As shown in FIG. 4b, the mimic significantly promoted Th2 differentiation whereas the antagomiR-21 inhibited Th2 differentiation, as measured by IL-4 and IL-5 secretion. The mimic and inhibitor did not significantly affect IFNγ, although there was a trend towards decreased IFNγ with both treatments. These data show for the first time that miR-21 can promote Th2 differentiation by a T cell autonomous mechanism.

The advantage of the miR-21 mimic is that it could better control the timing of miR-21 expression, and thus naïve wild-type CD4 T cells were treated with miR-21 mimic or scrambled control, and assessed downstream miR-21 targets after 12-16 hours of expression. A large number of miR-21 target genes have been described [36-39, 42], and six of the most well-known target mRNAs were analyzed (FIG. 8b). Of these six genes, only Sprouty1 (Spry1), a negative regulator of the MAP kinase pathway, was consistently decreased by mimic treatment (FIG. 4c). At the same time, the miR-21 mimic augmented Gata3 mRNA (FIG. 4c). These data indicate that miR-21 may augment GATA3 expression by decreasing Spry1 activity and up-regulating MAP kinase activity.

MiR-21 gene targets were next tested in wild-type versus Bcl6−/− Tregs (FIG. 4d). A decrease of Spry1 mRNA in was observed in Bcl6−/− Tregs compared to the wild-type Tregs, suggesting that the regulation of Spry1 by miR-21 occurs in Tregs. Additionally, a strong decrease in IL12a mRNA in Bcl6−/− Tregs was observed compared to the wild-type Tregs. IL12a is a component of the key Treg suppressive cytokine IL-35. These results suggest that the decrease in IL12a mRNA in Bcl6−/− Tregs may lead to less production of IL-35, which could contribute to the inability of Bcl6−/− Tregs to suppress Th2 inflammation [14, 43]. Thus, a critical target of miR-21 in Tregs appears to be an important immuno-suppressive cytokine. Next, to validate these miR-21 gene targets, these genes were assessed in the lungs of Ova-sensitized and intranasally challenged wild-type recipient mice (grey bars) or immunized i.p. with Bcl6−/− Tregs (black bars) or wild-type Tregs (white bars) mice (FIG. 4e). It was found that both Spry1 and IL12a mRNAs were significantly decreased in lungs of mice that received Bcl6−/− Tregs, whereas other miR-21 target mRNAs were not significantly decreased (FIG. 8c).

MiR-21 is a Biomarker for Th2-Type Eosinophilic Inflammation

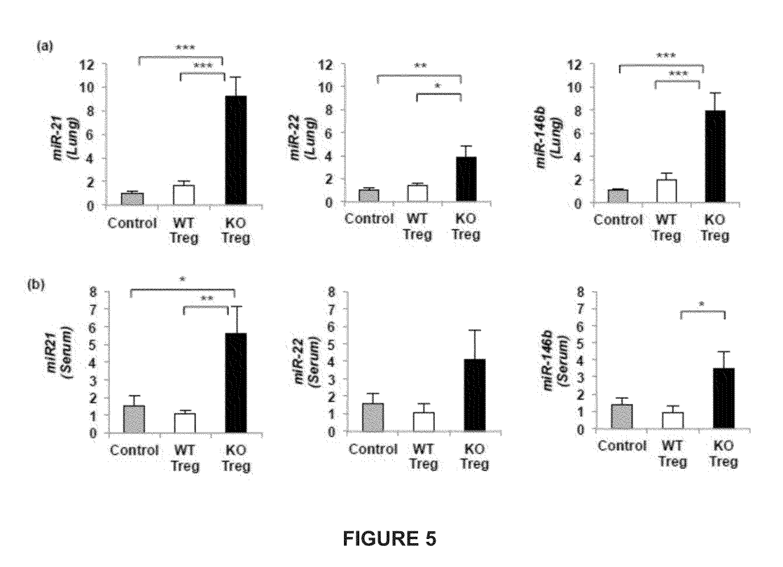

Since a decreased expression of miR-21 was observed in target genes in total lung that received Bcl6−/− Tregs, a test was conducted to establish whether miR-21 expression was increased in the lungs of the mice in which Th2 airway inflammation was induced. While miR-21 expression was not different between control and the wild-type Treg-treated mice, miR-21 was greatly increased in the total RNA of lungs taken from the Bcl6−/− Treg-treated mice (FIG. 5a). These data are consistent with decreased expression of miR-21 target genes in lungs that received Bcl6−/− Tregs, and also strongly support the idea that Bcl6−/− Tregs actively promote Th2 inflammation. While in a previous study, miR-146b was also seen up-regulated in Th2 inflammation, changes in miR-22 were not observed [38, 44]. In our model, miR-22 and miR-146b followed a similar pattern of expression in the lungs as miR-21 (FIG. 5a). These data indicate that up-regulation of miR-21, miR-22 and miR146b may represent a unique micro-RNA signature induced by Bcl6−/− Tregs in Th2-type inflammation.

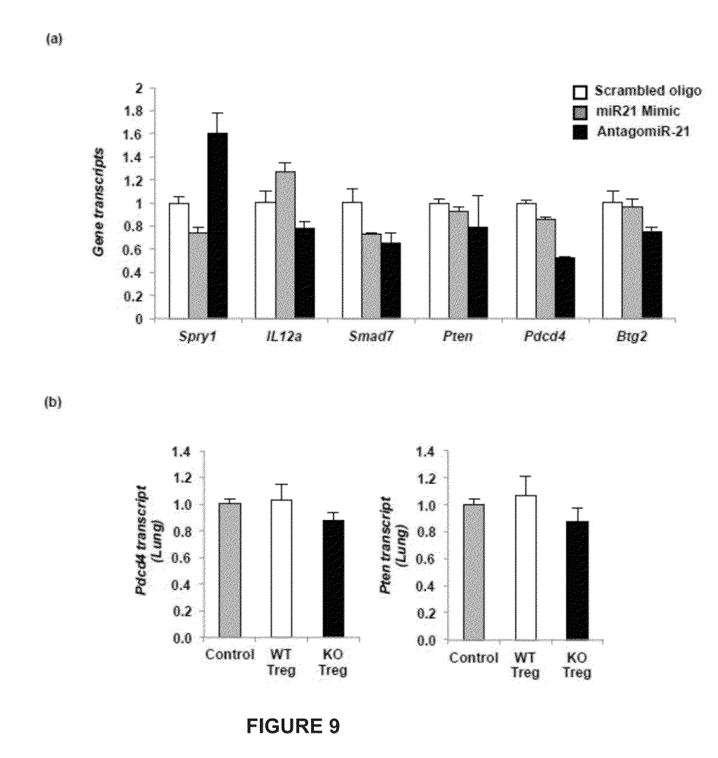

It was also found that the increase in miR-21 in the lungs that received Bcl6−/− Tregs correlated with circulating levels of miR-21, as has been seen in cancer [45-47]. Indeed, significantly elevated levels of miR-21 were found in serum samples from mice that received Bcl6−/− Tregs (FIG. 5b). Elevated serum levels of miR-22 and miR-146b were also found, though to a lesser degree than for miR-21. Nonetheless, these results indicate that increased serum miR-21 may represent a novel bio-marker for Th2-type inflammatory disease. It was next tested whether cell types other than Tregs expressed the high levels of miR-21 seen in mice that received Bcl6−/− Tregs. A previous study analyzing allergic airway inflammation showed that miR-21 was primarily expressed in myeloid cells in the inflamed lung [38]. As shown in FIG. 9, in situ hybridization was used to analyze miR-21 expression in the lungs of mice in the allergic airway inflammation model. While miR-21 expression was observed in myeloid cells in the inflammatory lesions of the lungs, the strongest expression of miR-21 was observed in the airway epithelium. Elevated expression of miR-21 in the total lungs of mice receiving Bcl6−/− Tregs correlated with more myeloid cells as well as higher expression of miR-21 in airway epithelium. These results indicate that an interplay between the Bcl6−/− Tregs and the airway epithelium leads to greater microRNA expression, which further correlates with increased amounts of circulating miR-21. The increased circulating microRNA is presumably due to exosomes shed from cells expressing these microRNAs [48, 49].

MicroRNA-21 is Elevated in the Sera of Pediatric Eosinophilic Esophagitis (EoE) and Asthma Patients

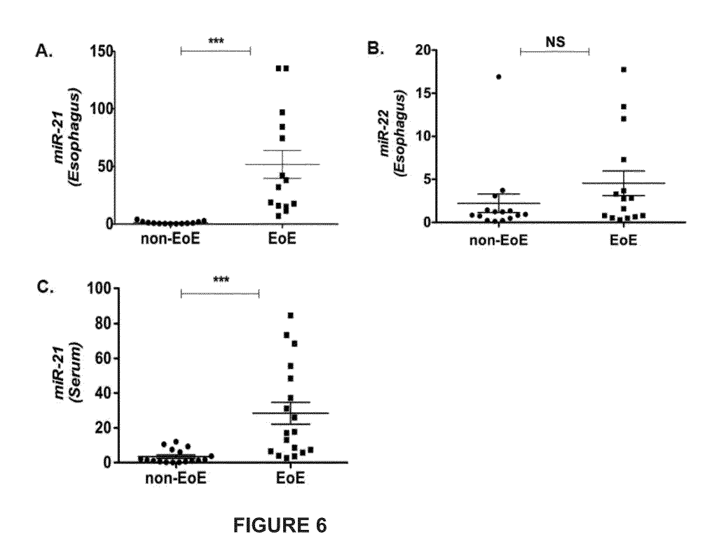

In order to shed further light on the role of miR-21 in Th2-type inflammation, it was initially tested whether miR-21 was increased in biopsy specimens from a randomly selected set of pediatric EoE patients [76]. It was found that, similar to Lu et al. [74] that miR-21 expression is increased in EoE biopsies compared to biopsies from control patients that had no detectable eosinophils (FIG. 6a). However, a greater fold elevation in miR-21 in EoE was observed, almost 50-fold on average, than that reported by Lu et al [74], who observed a roughly 4-fold increase in miR-21 in EoE. The expression of a different, randomly chosen microRNA, miR-22, was not significantly increased in EoE biopsies (FIG. 6b), showing that not all microRNAs are increased in EoE.

Because of the striking increase in miR-21 that was observed in esophageal biopsies of children with EoE, miR-21 in serum from EoE patients was assessed (FIG. 6c). Compared to controls, an average of 30-fold increase in serum miR-21 levels was observed in EoE patients. MiR-22 was not detected in the serum of EoE or control patients (data not shown). This miR-21 result contrasted with Lu et al. [74] who detected little to no levels of circulating miR-21 in EoE. Without being bound to any particular theory, some likely explanations for the differences between the above results and the Lu et al. report are: (1) possible differences in disease severity between patients in the two studies; (2) differences in control patients between the two studies, and (3) differences in assay sensitivity in detecting miR-21. Notably, Lu et al. compared circulating miR-21 in EoE patients to healthy atopic patients, whereas the controls in the EoE study reported herein were patients who had undergone esophagogastroduodenoscopy (EGD) for a variety of non-specific reasons but had normal histology on biopsies of the upper gastro-intestinal tract. Thus, unlike Lu et al., the non-EoE controls of the study reported herein were not enriched for atopy.

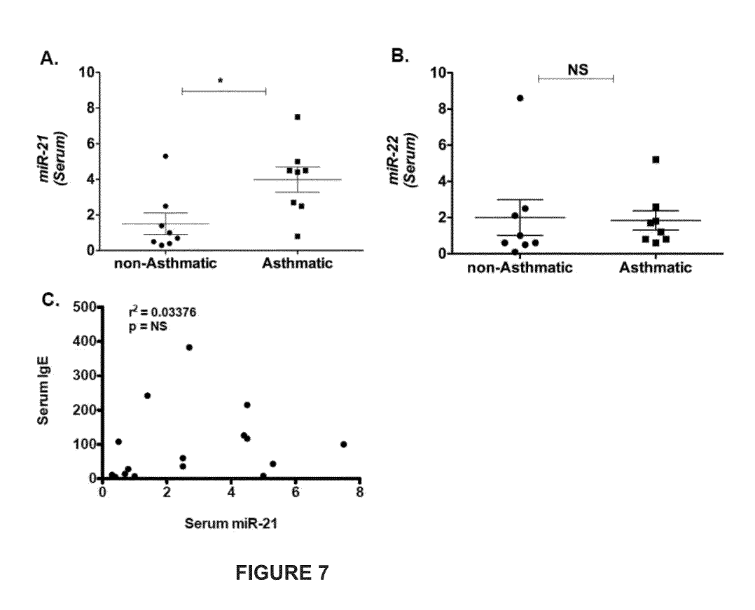

To better understand the relationship between atopy, allergic inflammatory disease and miR-21, serum miR-21 levels in pediatric patients recruited into a study on the development of asthma were analyzed. The patients were recruited as infants, based on a diagnosis of dermatitis, and subsequently characterized for atopic status and the development of asthma [77]. Atopic status was defined as the presence of specific IgE to at least 1 out of 10 allergens tested. It was found that in a randomly selected set of 16 patients, tested at 5 years of age, serum miR-21 increased in the asthma patients by 4-fold, with a p value of 0.018 (FIG. 7a). MiR-22 showed no increase in patients with asthma (FIG. 7b). In the selected patients, 8 out of 16 patients were atopic, however atopy was randomly distributed between asthmatic and non-asthmatic patients. While asthmatic patients had on average a two-fold increase in serum IgE over non-asthmatic patients (130 IU/ml versus 60 IU/ml), the difference in average IgE was not significant (p=0.24). MiR-21 levels did not associate positively or negatively with atopy in this set of patients (FIG. 7c). Thus, serum miR-21 is a novel biomarker for asthma, independent of atopy. Overall, these results indicate that elevated circulating miR-21 can serve as a biomarker for allergic inflammatory diseases such as EoE and asthma. While it was found that EoE patients have a much greater increase in serum miR-21 than asthmatic patients, this difference may relate to the severity of disease at the time of sample collection.

### Materials and Methods

Mice. Bcl6−/− mice on a mixed C57BL/6-129Sv background have been previously described [1, 2]. Foxp3-gfp mice were obtained from the Jackson Laboratory (Bar Harbor, Me.) and Bcl6−/− mice were mated onto the Foxp3-gfp background. Rag1−/−, IL-10−/− and C57BL/6 congenic CD45.1⁺ (BoyJ) wild-type mice were also originally obtained from Jackson labs. The wild-type BoyJ mice were bred onto the Foxp3-gfp background. Bcl6−/− Tcrα−/− mice have been previously described 53. Wild-type (WT) and Bcl6−/− Foxp3-gfp mice were genotyped by PCR as previously described. Mice were bred under specific pathogen-free conditions at the laboratory animal facility at Indiana University School of Medicine (IUSM) and were handled according to protocols approved by the IUSM Animal Use and Care Committee. Healthy control and EE patient sample collection and analysis were approved by the Institutional Review Board of Indiana University and required parental consent for samples from infants.

Antibodies and FACS analysis. Flow cytometry analysis of intracellular transcription factors and cytokines was performed by staining the cells with fluorochrome-conjugated anti-FoxP3 (FJK-16a; eBioscience), anti-Helios (22F6; BioLegend), anti-GATA3 (TWAJ; eBioscience) and anti-IL-4 (BD Biosciences) using the mouse Regulatory T cell staining kit (eBioscience). Cells were first stained with antibodies for the desired cell surface markers—CD4 (RM4-5; BD Biosciences) and CD25 (PC61.5; eBioscience), followed by permeabilization with Fixation/Permeabilization buffer and intracellular staining in Permeabilization buffer. For the bone marrow chimera experiments, cells were stained for Treg markers (CD4, CD25) and CD45.1 (A20; BD Biosciences). Flow analysis was performed on a FACSCalibur and data analyzed using the CellQuest software (Becton Dickinson).

Mouse Thelper cell differentiation and Treg cell assays. Naïve T cells (CD4⁺CD62L⁺) were purified from lymph nodes and spleen using magnetic beads (Miltenyi Biotech). Naïve CD4⁺ T cells (1×106 cells/ml in DMEM medium supplemented with 10% FCS (Atlanta Biologicals), 2 mM glutamine, 100 units/ml Penicillin-Streptomycin, MEM nonessential amino acids, 25 mM HEPES and 55 uM 2-mercaptoethanol (Gibco)) were activated with plate-bound anti-CD3 (5 μg/ml; 145-2C11; BD Biosciences) and anti-CD28 (10 μg/ml; 37.51; BD Biosciences) and polarized under Th0 (with no added cytokines) and Th2 (20 ng/ml IL-4 and 10 μg/ml anti-IFNγ) differentiation conditions. Recombinant mouse IL-4 was purchased from R&D Systems and Abs to CD3 and CD28 from BD Biosciences. Recombinant human IL-2 was obtained from the Biological Resources Branch, Division of Cancer Treatment and Diagnosis, National Cancer Institute-Frederick Cancer Research and Development Center.

For Treg assays, CD4⁺CD25⁺ T cells were purified from wild-type and Bcl6−/− Foxp3-gfp mice using magnetic beads, followed by FACS sorting for pure GFP⁺ Tregs using FACSAria cell sorter (Becton Dickinson). The CD4⁺CD25⁻ T cell fraction from magnetic bead isolation was used as responder T cells (Tresps). Antigen-presenting cells (APCs) were prepared from the spleens of TCRα−/− mice. For Treg suppression assays, wild-type or Bcl6−/− Tresps (50×103/well) were separately co-cultured with wild-type or Bcl6−/− Tregs at different ratios as indicated along-with Mitomycin-C (20 μg/ml) treated APCs (50×103/well) plus anti-CD3 (2 μg/ml) or 10 μg/ml Ova for 72 hours. Plates were pulsed with 1 μC of [³H] thymidine at 48 hrs of culture and cell proliferation in triplicate cultures was measured using a scintillation counter.

Induction of experimental colitis. Un-fractionated CD4⁺ T cells (Tresps) (1×106) from IL10−/− mice isolated using magnetic bead selection were adoptively transferred intraperitoneally (i.p.) into Rag−/− mice to induce a strong Th1-mediated colitogenic response in the recipients 4-8 weeks post-transfer [29]. Along with the transfer of IL10−/− Tresps, two cohorts of Rag−/− recipients were injected i.p. with FACS-sorted CD4⁺CD25⁺Foxp3⁺ Tregs (250×103/mouse) from Bcl6−/− or wild-type Foxp3gfp mice. The recipient mice were monitored for signs of intestinal pathology and weight loss over a period of 4-5 weeks, following which mice were sacrificed to assess severity of colitis using the parameters of percent weight loss, changes in colon length and colon histology scores. Colon sections were stained with hematoxylin and eosin (H&E) and colitis severity was graded in a blinded fashion on a scale of 0-6 as described: 0—Normal crypt architecture and occasional cell infiltration, 2—Irregular crypt architecture and increasing number of cells in lamina propria (LP), 4—Moderate crypt loss (10-50%) and confluence of cells extending to sub-mucosa, 6—Severe crypt loss (50-90%) and transmural extension of infiltrate.

Induction of experimental allergic airway inflammation. Both wild-type B6 female mice (recipients) as well as the Bcl6−/− and wild-type Foxp3-gfp mice (Treg donors) were sensitized intra-peritoneally (i.p.) with Ova (Sigma) adsorbed to alum (Sigma) at a dose of 20 μg Ova/2 mg alum on days 0 and 7 of the protocol [30]. On day 14, CD4⁺CD25⁺Foxp3⁺ Tregs (350×103 cells/mouse) were FACS-sorted from Ova-sensitized Bcl6−/− and wild-type Foxp3-gfp mice and then injected i.p. into the sensitized wild-type B6 female recipients [31]. After 3 hours following immunization, recipient mice were then challenged intranasally with Ova for 5 consecutive days (100 μg/day). Mice were sacrificed by i.p. injection of pentobarbital (5 mg/mouse) 48 hours after the final intranasal challenge. The trachea was cannulated and lungs were lavaged three times with 1 ml PBS to collect the bronchioalveolar lavage (BAL). Cells recovered in BAL fluid and the lung mediastinal lymph nodes (MLNs) were counted with a hemocytometer. Eosinophils, neutrophils, T cells, B cells and mononuclear cells in the BAL fluid were distinguished by cell size and by expression of CD3, B220, CCR3, CD11c and major histocompatibility complex class II, analyzed by flow cytometry as described [30]. For quantitative PCR analysis, lung tissues were homogenized in a tissue lyser (Qiagen) and RNA isolated with an RNeasy kit (Qiagen) was used for synthesis of cDNA for subsequent analysis. Paraffin-embedded sections were stained with H & E for evaluation of the infiltration of inflammatory cells by light microscopy.

Airway hyper-reactivity to methacholine challenge was determined 24 hours after the final intranasal challenge. Noninvasive unrestrained whole-body plethysmography (Buxco Research Systems) was used to record airway responsiveness with the dimensionless parameter ‘enhanced pause’ for estimation of total pulmonary resistance, an indicator of broncho-constriction. Mice were placed in whole-body plethysmographs and baseline measurements were recorded. Saline was administered by nebulization for 2 minutes, followed by increasing doses of methacholine, and the enhanced-pause parameter was recorded over 5 minutes.

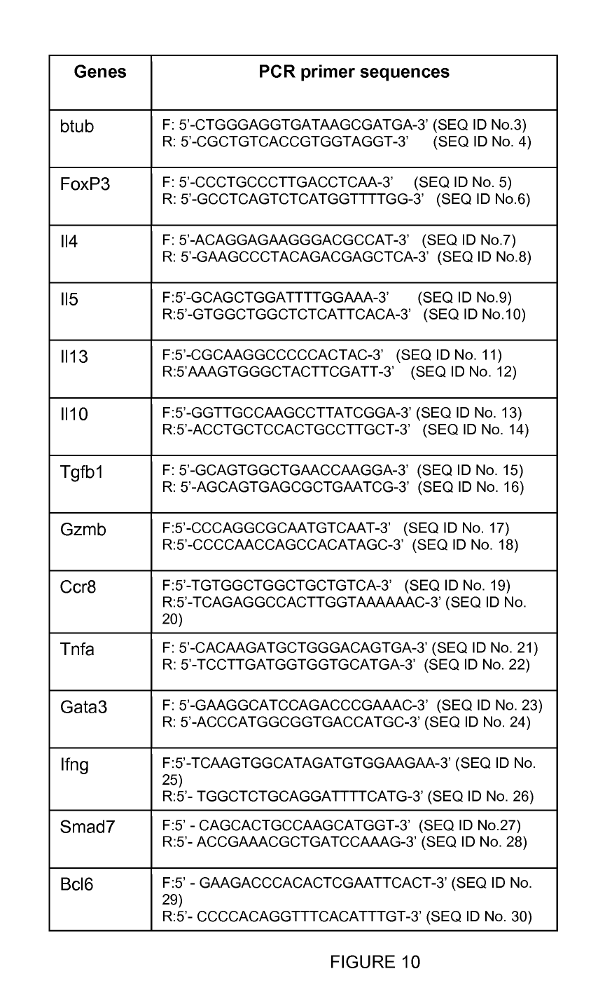

Affymetrix Microarrays and qRT-PCR. Total RNA was extracted from FACS-sorted CD4⁺CD25⁺FoxP3⁺ Tregs from Bcl6−/− and wild-type Foxp3-gfp mice following 16 hours activation in vitro with plate-bound anti-CD3 (5 μg/ml) and anti-CD28 (10 μg/ml) using the RNeasy Mini kit, according to the manufacturer's protocol (Qiagen). The microarray studies were carried out using the facilities of the Center for Medical Genomics at Indiana University School of Medicine which process the samples employing the protocols recommended by Affymetrix in their GeneChip® Expression Analysis Technical Manual (Affymetrix, Santa Clara, Calif.). Biotinylated cRNA was hybridized to Affymetrix GeneChip Mouse Genome 430 2.0 arrays. Data analysis was performed using the MeV software. To validate the expression array data, qPCR was performed on independently prepared Tregs from Bcl6−/− and wild-type Foxp3-gfp mice. PCR primer sequences are listed in Table 1 (FIG. 10).

Retroviral transductions. Naïve T cells (CD4⁺CD62L⁺) prepared from wild-type C57BL/6 or Stat6−/− mice using magnetic beads were activated in vitro with plate-bound anti-CD3 (5 μg/ml) and anti-CD28 (10 μg/ml) for 24 hrs. Cells were then transduced by spin infection with bicistronic retroviral vector (RV) supernatants encoding Bcl6 and H2Kk or GATA3 and hCD48, 68. Naïve T cells activated similarly under Th0 and Th2 differentiation conditions were infected with retroviral supernatant encoding miR-21 and H2Kk. Second or third day following transduction, cells infected with H2Kk RVs were stained with biotin-anti-H2Kk and streptavidin-APC, while those infected with hCD4 RVs were stained for anti-hCD4-PE and then FACS-sorted based on APC or PE expression, respectively. The sorted RV⁺ T cells were re-stimulated in vitro with anti-CD3 and anti-CD28 for 4-6 hours for gene expression analysis or overnight for cytokine measurements.

Overexpression and inhibition of miR-21. Naïve T cells activated under Th0 conditions (with nothing added) were treated with 1 μM control (scrambled oligo), miR21 mimic (double stranded RNA oligo) and antagomiR (single stranded DNA oligo) (Exiqon). The oligos were cholesterol linked that enabled efficient delivery into cells, without the need of any transfection protocol. Gene expression was assessed 12-16 hours following treatment. For assessment of cytokine productions, cells were treated with the oligos over a 5-day period, following which the cultures were re-stimulated with anti-CD3 (10 ug/ml) overnight to obtain cell-free supernatants for ELISA.

Reporter assays. Jurkat T cells (10×106 cells/250 ul) were electroporated as described [69] in serum-free RPMI 1640 medium with an IL-5 promoter-driven luciferase reporter vector (10 ug) along with expression constructs (10 ug) for CXN, CXN-GATA3, CXNBCL6 or CXN-GATA3 plus CXN-BCL6 at the concentrations listed. After electroporation, cells were re-suspended in RPMI media supplemented with 10% FCS and rested overnight. For experiments with co-transfection of full-length, SB1 and SB2 miR21 promoter constructs (1 ug) and expression constructs for CXN or CXN-BCL6 (1 ug), 1×106 cells Jurkat T cells were transiently transfected with X-tremeGENE HP DNA transfection reagent (Roche), according to the manufacturer's protocol. Luciferase measurements were performed 24 hours after electroporation or transfection following 6 hours activation of cells with PMA (10 ng/ml) and lonomycin (0.3 μM) using Luciferase Assay System (Promega). M12 B cells were electroporated as described [70] with the indicated plasmids as above. Luciferase measurements were performed following activation of cells with PMA and dibutyrl cAMP.

MicroRNA profiling and qRT-PCR assessment of microRNAs. RNA was extracted from FACS-sorted CD4⁺CD25⁺Foxp3⁺ Tregs from wild-type and Bcl6−/− Foxp3-gfp mice following 16 hours activation in vitro with anti-CD3 (5 μg/ml) and anti-CD28 (10 μg/ml) using MiRNeasy Mini kit, according to manufacturer's protocol (Qiagen). The quality of the total RNA was verified by an Agilent 2100 Bioanalyzer profile. 140 ng total RNA was labeled with fluorescent label. The samples were hybridized to the miRCURY™ LNA array version 11.0 (Exiqon, Denmark), which contains capture probes targeting all miRNAs for human, mouse or rat registered in the miRBASE version 13.0 at the Sanger Institute.

Validation of the differentially expressed microRNAs from the microarray dataset, assessment of microRNA expression profile of the total lungs and esophageal biopsies as well as from mouse and human serum was performed using TaqMan microRNA assays (Applied Biosystems), according to the manufacturer's protocol. Normalization was performed using sno202, sno234 and U6 as controls, with U6 as the sole control for samples with limiting RNA. Quantitative real-time PCR was performed by assaying each sample in triplicates, including no-template controls, on a Stratagene Mx3000P real-time PCR system. Relative expression was calculated using the delta-delta CT (ddCT) method, as previously described.

Generation of Bone Marrow Chimeras. Donor wild-type BoyJ (CD45.1⁺) and Bcl6−/− (CD45.1⁻) Foxp3-gfp mice were euthanized with CO₂ asphyxiation and cervical dislocation, and femurs and tibias were removed aseptically. Bone marrow (BM) was flushed with DMEM complete media. Recipient Rag−/− mice were sub-lethally irradiate (350 Gy) 16-24 hours prior to re-constitution. The recipients were then re-constituted with wild-type or Bcl6−/− BM cells (10×106) by intravenous injections (i.v.). Satisfactory reconstitution was achieved after 4-5 months, following which the mice were sacrificed and wild-type and Bcl6−/− CD25⁺FoxP3⁺ (Tregs) and CD25-FoxP3⁻ (Tresps) were sorted based on CD45.1 expression for quantitative PCR analysis.

Cloning of mmu-microRNA-21 and plasmid construction for miR-21 reporter vectors. The miR-21 gene representing the primary transcript (˜300 bp) was PCR amplified from mouse genomic DNA and cloned into retroviral vector co-expressing H2Kk via EcoRI (restriction enzyme). The microRNA is expressed as a partial-primary microRNA transcript, transcribed from the retroviral LTR.

F (miR-21): 5′-AATT-GAATTC-GGTACC-TTGGCATTAAGCCCCAGCAAACC-3′
R (miR-21): 5′-AATT-GAATTC-TCCAAGTCTCACAAGACATAAGGACC-3′

Full-length mouse miR-21 promoter region was PCR amplified from mouse genomic DNA and was inserted using Mlu-I and Xho-I restriction enzyme sites into the pGL3-basic vector (Promega). SB1 and SB2 mutations in the miR-21 promoter region were created by insertion of restriction enzyme sites in the respective STAT/BCL6 binding site by amplifying with mutated primer sequences, followed by insertion into the pGL3-basic vector. Primer sequences listed in Table 2. All constructs were sequenced prior to their application to confirm construct integrity.

In situ hybridization. In situ hybridizations were performed in 8 μM cryosections from lungs of mice in the airway inflammation experiment using the miRCURY LNA microRNA ISH Optimization kit 2 (miR-21), according to the manufacturer's protocol (Exiqon). In brief, the FFPE slides were rinsed and digested with Proteinase K for 12 minutes at 37° C. After protease digestion, the digoxin-labeled LNA-scrambled control probe and LNA miR-21 antisense probe (Exiqon) were hybridized to the slides at 52° C. for 6 hours. Following post-hybridization washes with SSC buffer at 47° C., 100 ul of rabbit antidigoxin (Sigma-Aldrich) Ab, diluted 1/2000 was applied to the slides for 1 hour at room temperature. The slides were rinsed and then incubated with 100 ul anti-rabbit alkaline phosphatase and TNBS substrate for 2 hour at 30° C. Slides were counterstained with Nuclear Fast Red (Polyscientific), coverslipped, and mounted for viewing.

Cytokine secretion analysis. Cytokines were measured from 24 hour activated cell-free supernatants by ELISA. IL-4, IL-5, IFNγ and IL-17 ELISA reagents were purchased from BD Biosciences; IL-13 reagents purchased from R&D systems.

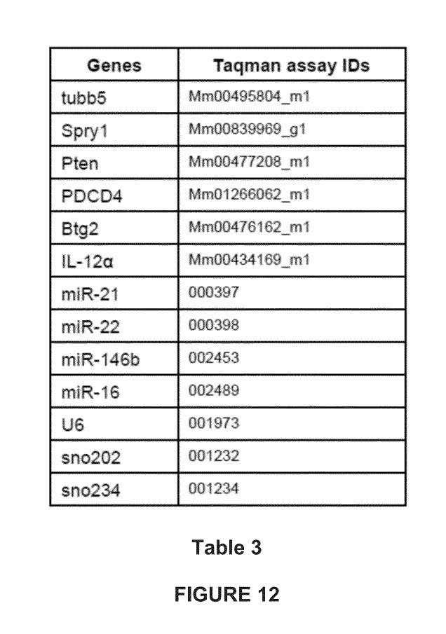

Gene expression analysis. Total cellular RNA was prepared using the Trizol method (Life Technologies), and cDNA prepared with the Transcriptor First Strand cDNA synthesis kit (Roche). Quantitative PCR reactions were run by assaying each sample in triplicates using the Fast Start Universal SYBR Green Mix (Roche Applied Science) with a Stratagene Mx3000P Real-Time QPCR machine. Samples with limiting RNA were assessed for gene expression using Taqman assays (ABI). Levels of mRNA expression were normalized to beta-tubulin mRNA levels, and differences between samples analyzed using the ddCT method. PCR primer sequences are listed in Table 1 and Taqman assay IDs are listed in Table 3 (FIG. 12).

Statistical analysis. p values were calculated using Students' T-test. A p value<0.05 was considered to show a significant difference.

Assessment of microRNA expression of the esophageal biopsies as well as from serum was performed using TaqMan microRNA assays (Applied Biosystems), according to the manufacturer's protocol. Normalization was performed using sno202, sno234 and U6 as controls, with U6 as the sole control for samples with limiting RNA. qPCR was performed by assaying each sample in triplicates, including no-template controls, on a Stratagene Mx3000P real-time PCR system. Relative expression was calculated using the delta-delta CT (ddCT) method.

### REFERENCES

1. Dent, A. L., Hu-Li, J., Paul, W. E. & Staudt, L. S. T helper type 2 inflammatory disease in the absence of IL-4 and STAT6. Proc Natl Acad Sci USA 95, 13823-13828 (1998).
2. Dent, A. L., Shaffer, A. L., Yu, X., Allman, D. & Staudt, L. M. Control of inflammation, cytokine expression, and germinal center formation by BCL-6. Science 276, 589-592 (1997).
3. Ye, B. H. et al. The BCL-6 proto-oncogene controls germinal-centre formation and Th2-type inflammation. Nat Genet. 16, 161-170 (1997).
4. Kusam, S., Toney, L. M., Sato, H. & Dent, A. L. Inhibition of Th2 differentiation and GATA-3 expression by BCL-6. J Immunol 170, 2435-2441 (2003).
5. Johnston, R. J. et al. Bcl6 and Blimp-1 are reciprocal and antagonistic regulators of T follicular helper cell differentiation. Science 325, 1006-1010 (2009).
6. Nurieva, R. I. et al. Bcl6 mediates the development of T follicular helper cells. Science 325, 1001-1005 (2009).
7. Yu, D. et al. The Transcriptional Repressor Bcl-6 Directs T Follicular Helper CellLineage Commitment. Immunity (2009).
8. Mondal, A., Sawant, D. & Dent, A. L. Transcriptional repressor BCL6 controls Th17 responses by controlling gene expression in both T cells and macrophages. J Immunol 184, 4123-4132 (2010).
9. Hori, S., Nomura, T. & Sakaguchi, S. Control of regulatory T cell development by the transcription factor Foxp3. Science 299, 1057-1061 (2003).
10. Fontenot, J. D., Gavin, M. A. & Rudensky, A. Y. Foxp3 programs the development and function of CD4+CD25+ regulatory T cells. Nat Immunol 4, 330-336 (2003).
11. Fontenot, J. D. et al. Regulatory T cell lineage specification by the forkhead transcription factor foxp3. Immunity 22, 329-341 (2005).
12. Pandiyan, P., Zheng, L., Ishihara, S., Reed, J. & Lenardo, M. J. CD4⁺CD25⁺Foxp3⁺ regulatory T cells induce cytokine deprivation-mediated apoptosis of effector CD4+ T cells. Nat Immunol 8, 1353-1362 (2007).
13. Asseman, C., Mauze, S., Leach, M. W., Coffman, R. L. & Powrie, F. An essential role for interleukin 10 in the function of regulatory T cells that inhibit intestinal inflammation. J Exp Med 190, 995-1004 (1999).
14. Collison, L. W. et al. The inhibitory cytokine IL-35 contributes to regulatory Tcell function. Nature 450, 566-569 (2007).
15. Li, M. O., Wan, Y. Y. & Flavell, R. A. T cell-produced transforming growth factorbeta1 controls T cell tolerance and regulates Th1- and Th17-cell differentiation. Immunity 26, 579-591 (2007).
16. Liang, B. et al. Regulatory T cells inhibit dendritic cells by lymphocyte activation gene-3 engagement of MHC class II. J Immunol 180, 5916-5926 (2008).
17. Fallarino, F. et al. Modulation of tryptophan catabolism by regulatory T cells. Nat Immunol 4, 1206-1212 (2003).
18. Grossman, W. J. et al. Differential expression of granzymes A and B in humancytotoxic lymphocyte subsets and T regulatory cells. Blood 104, 2840-2848 (2004).
19. Cao, X. et al. Granzyme B and perforin are important for regulatory T cell-mediated suppression of tumor clearance. Immunity 27, 635-646 (2007).
20. Koch, M. A. et al. The transcription factor T-bet controls regulatory T cell homeostasis and function during type 1 inflammation. Nat Immunol 10, 595-602 (2009).
21. Zheng, Y. et al. Regulatory T-cell suppressor program co-opts transcription factor IRF4 to control T(H)2 responses. Nature 458, 351-356 (2009).
22. Chaudhry, A. et al. CD4+ regulatory T cells control TH17 responses in a Stat3-dependent manner. Science 326, 986-991 (2009).
23. Godfrey, V. L., Wilkinson, J. E. & Russell, L. B. X-linked lymphoreticular disease in the scurfy (sf) mutant mouse. Am J Pathol 138, 1379-1387 (1991).
24. Tivol, E. A. et al. Loss of CTLA-4 leads to massive lymphoproliferation and fatal multiorgan tissue destruction, revealing a critical negative regulatory role of CTLA-4. Immunity 3, 541-547 (1995).
25. Kulkarni, A. B. et al. Transforming growth factor beta 1 null mutation in mice causes excessive inflammatory response and early death. Proc Natl Acad Sci USA 90, 770-774 (1993).
26. Linterman, M. A. et al. Foxp3(+) follicular regulatory T cells control the germinal center response. Nat Med 17, 975-982 (2011).
27. Chung, Y. et al. Follicular regulatory T cells expressing Foxp3 and Bcl-6 suppress germinal center reactions. Nat Med 17, 983-988 (2011).
28. Thornton, A. M. et al. Expression of Helios, an Ikaros transcription factor family member, differentiates thymic-derived from peripherally induced Foxp3⁺ T regulatory cells. J Immunol 184, 3433-3441 (2010).
29. Ostanin, D. V. et al. T cell transfer model of chronic colitis: concepts, considerations, and tricks of the trade. Am J Physiol Gastrointest Liver Physiol 296, G135-146 (2009).
30. Chang, H. C. et al. The transcription factor PU.1 is required for the development of IL-9-producing T cells and allergic inflammation. Nat Immunol 11, 527-534 (2010).
31. Presser, K. et al. Coexpression of TGF-beta1 and IL-10 enables regulatory T cells to completely suppress airway hyperreactivity. J Immunol 181, 7751-7758 (2008).
32. Yoshida, K. et al. Bcl6 controls granzyme B expression in effector CD8⁺ T cells. Eur J Immunol 36, 3146-3156 (2006).
33. Ouyang, W. et al. Stat6-Independent GATA-3 Autoactivation Directs IL-4-Independent Th2 Development and Commitment. Immunity 12, 27-37 (2000).
34. Zhou, X. et al. Selective miRNA disruption in T reg cells leads to uncontrolled autoimmunity. J Exp Med 205, 1983-1991 (2008).
35. Liston, A., Lu, L. F., O'Carroll, D., Tarakhovsky, A. & Rudensky, A. Y. Dicer-dependent microRNA pathway safeguards regulatory T cell function. J Exp Med 205, 1993-2004 (2008).
36. Jung, E. J. & Calin, G. A. The Meaning of 21 in the MicroRNA world: perfection rather than destruction? Cancer Cell 18, 203-205 (2010).
37. Thum, T. et al. MicroRNA-21 contributes to myocardial disease by stimulating MAP kinase signalling in fibroblasts. Nature 456, 980-984 (2008).
38. Lu, T. X., Munitz, A. & Rothenberg, M. E. MicroRNA-21 is up-regulated in allergic airway inflammation and regulates IL-12p35 expression. J Immunol 182, 4994-5002 (2009).
39. Fujita, S. et al. miR-21 Gene expression triggered by AP-1 is sustained through a double-negative feedback mechanism. J Mol Biol 378, 492-504 (2008).
40. Loffler, D. et al. Interleukin-6 dependent survival of multiple myeloma cells involves the Stat3-mediated induction of microRNA-21 through a highly conserved enhancer. Blood 110, 1330-1333 (2007).
41. van der Fits, L. et al. MicroRNA-21 expression in CD4⁺ T cells is regulated by STAT3 and is pathologically involved in Sezary syndrome. J Invest Dermatol 131, 762-768 (2011).
42. Hatley, M. E. et al. Modulation of K-Ras-dependent lung tumorigenesis by MicroRNA-21. Cancer Cell 18, 282-293 (2010).
43. Huang, C. H. et al. Airway Inflammation and IgE Production Induced by Dust Mite Allergen-Specific Memory/Effector Th2 Cell Line Can Be Effectively Attenuated by IL-35. J Immunol 187, 462-471 (2011).
44. Garbacki, N. et al. MicroRNAs profiling in murine models of acute and chronic asthma: a relationship with mRNAs targets. PLoS One 6, e16509 (2010).
45. Wei, J. et al. Identification of plasma microRNA-21 as a biomarker for early detection and chemosensitivity of non-small cell lung cancer. Chin J Cancer 30, 407-414 (2011).
46. Tomimaru, Y. et al. Circulating microRNA-21 as a novel biomarker for hepatocellular carcinoma. J Hepatol (2011).
47. Asaga, S. et al. Direct serum assay for microRNA-21 concentrations in early and advanced breast cancer. Clin Chem 57, 84-91 (2010).
48. Hunter, M. P. et al. Detection of microRNA expression in human peripheral blood microvesicles. PLoS One 3, e3694 (2008).
49. Taylor, D. D. & Gercel-Taylor, C. MicroRNA signatures of tumor-derived exosomes as diagnostic biomarkers of ovarian cancer. Gynecol Oncol 110, 13-21 (2008).
50. Liacouras, C. A. et al. Eosinophilic esophagitis: Updated consensus recommendations for children and adults. J Allergy Clin Immunol 128, 3-20 e26 (2011).
51. Noel, R. J. & Rothenberg, M. E. Eosinophilic esophagitis. Curr Opin Pediatr 17, 690-694 (2005).
52. Rothenberg, M. E. Biology and treatment of eosinophilic esophagitis. Gastroenterology 137, 1238-1249 (2009).
53. Toney, L. M. et al. BCL-6 regulates chemokine gene transcription in macrophages. Nat. Immunol. 1, 214-220 (2000).
54. Yoshida, T. et al. The role of Bcl6 in mature cardiac myocytes. Cardiovasc Res 42, 670-679 (1999).
55. Wan, Y. Y. & Flavell, R. A. Regulatory T-cell functions are subverted and converted owing to attenuated Foxp3 expression. Nature 445, 766-770 (2007).
56. Hatley, M. E. et al. Modulation of K-Ras-dependent lung tumorigenesis by MicroRNA-21. Cancer Cell 18, 282-293.
57. Yamashita, M. et al. Ras-ERK MAPK cascade regulates GATA3 stability and Th2 differentiation through ubiquitin-proteasome pathway. J Biol Chem 280, 29409-29419 (2005).
58. Iliopoulos, D., Jaeger, S. A., Hirsch, H. A., Bulyk, M. L. & Struhl, K. STAT3 activation of miR-21 and miR-181b-1 via PTEN and CYLD are part of the epigenetic switch linking inflammation to cancer. Mol Cell 39, 493-506 (2011).
59. Liu, G. et al. miR-21 mediates fibrogenic activation of pulmonary fibroblasts and lung fibrosis. J Exp Med 207, 1589-1597 (2010).
60. Si, M. L. et al. miR-21-mediated tumor growth. Oncogene 26, 2799-2803 (2007).
61. Stagakis, E. et al. Identification of novel microRNA signatures linked to human lupus disease activity and pathogenesis: miR-21 regulates aberrant T cell responses through regulation of PDCD4 expression. Ann Rheum Dis 70, 1496-1506.
62. Pan, W. et al. MicroRNA-21 and microRNA-148a contribute to DNA hypomethylation in lupus CD4+ T cells by directly and indirectly targeting DNA methyltransferase 1. J Immunol 184, 6773-6781 (2010).
63. Wu, F. et al. MicroRNAs are differentially expressed in ulcerative colitis and alter expression of macrophage inflammatory peptide-2 alpha. Gastroenterology 135, 1624-1635 e1624 (2008).
64. Jeker, J. & Bluestone, J. A. Small RNA regulators of T cell-mediated autoimmunity. J Clin Immunol 30, 347-357 (2010).
65. Lu, L. F. et al. Function of miR-146a in controlling Treg cell-mediated regulation of Th1 responses. Cell 142, 914-929 (2011).
66. Sheedy, F. J. et al. Negative regulation of TLR4 via targeting of the proinflammatory tumor suppressor PDCD4 by the microRNA miR-21. Nat Immunol 11, 141-147 (2009).
67. Rouas, R. et al. Human natural Treg microRNA signature: role of microRNA-31 and microRNA-21 in FOXP3 expression. Eur J Immunol 39, 1608-1618 (2009).
68. Chang, H. C. et al. PU.1 Expression Delineates Heterogeneity in Primary Th2 Cells. Immunity 22, 693-703 (2005).
69. Vasanwala, F. H., Kusam, S., Toney, L. M. & Dent, A. L. Repression of AP-1 function: a mechanism for the regulation of Blimp-1 expression and B-lymphocyte differentiation by the B cell lymphoma-6 protooncogene. J Immunol 169, 1922-1929 (2002).
70. Zhang, D. H., Yang, L. & Ray, A. Differential responsiveness of the IL-5 and IL-4 genes to transcription factor GATA-3. J Immunol 161, 3817-3821 (1998).
71. Cho W C. OncomiRs: the discovery and progress of microRNAs in cancers. Mol Cancer 2007; 6:60.
72. Jeker J, Bluestone J A. Small RNA regulators of T cell-mediated autoimmunity. J Clin Immunol 2010; 30:347-57.
73. Lu T X, Munitz A, Rothenberg M E. MicroRNA-21 is up-regulated in allergic airway inflammation and regulates IL-12p35 expression. J Immunol 2009; 182:4994-5002.
74. Lu T X, Sherrill J D, Wen T, Plassard A J, Besse J A, Abonia J P, et al. MicroRNA signature in patients with eosinophilic esophagitis, reversibility with glucocorticoids, and assessment as disease biomarkers. J Allergy Clin Immunol 2012; 129:1064-75 e9.
75. Lu T X, Hartner J, Lim E J, Fabry V, Mingler M K, Cole E T, et al. MicroRNA-21 limits in vivo immune response-mediated activation of the IL-12/IFN-gamma pathway, Th1 polarization, and the severity of delayed-type hypersensitivity. J Immunol 2011; 187:3362-73.
76. Subbarao G, Rosenman M B, Ohnuki L, Georgelas A, Davis M, Fitzgerald J F, et al. Exploring potential noninvasive biomarkers in eosinophilic esophagitis in children. Pediatr Gastroenterol Nutr 2011; 53:651-8.
77. Tepper R S, Llapur C J, Jones M H, Tiller C, Coates C, Kimmel R, et al. Expired nitric oxide and airway reactivity in infants at risk for asthma. J Allergy Clin Immunol 2008; 122:760-5.

1. A method of screening for a Th2 inflammatory disease in a subject, the method comprising the steps of:
assaying miR-21 expression level in a biological sample from the subject by at least one of quantitative PCR, a microarray, or in situ hybridization; and
comparing miR-21 expression level in the biological sample from the subject to miR-21 expression level in a control; and
determining the subject has a Th2 inflammatory disease if there is an increase in miR-21 expression level in the biological sample from the subject compared to miR-21 expression level in the control.

2. The method of claim 1, further comprising the step of developing a treatment plan if the increase in miR-21 expression level in the biological sample from the subject is at least 5-fold compared to miR-21 expression level in the control.

3. The method of claim 1, wherein the biological sample is selected from the group consisting of blood, serum, a biopsy sample, a tissue sample, a cell suspension, saliva, oral fluid, cerebrospinal fluid, lymph, urine, gastric fluid, synovial fluid, mucus, sputum, and mixtures thereof.

4. The method of claim 1, wherein the disease is selected from the group consisting of allergic asthma, eosinophilic esophagitis, allergic rhinitis, atopic dermatitis, and combinations thereof.

5. The method of claim 1, wherein assaying miR-21 expression level in the biological sample from the subject is by quantitative PCR.

6. The method of claim 1, wherein assaying miR-21 expression level in the biological sample from the subject is by a microarray.

7. The method of claim 1, wherein assaying miR-21 expression level in the biological sample from the subject is by in situ hybridization

8. The method of claim 1, further comprising assaying miR-22 in a sample from the subject.

9. The method of claim 1, further comprising assaying miR-146b in a sample from the subject.

10. A method for screening for eosinophilic esophagitis in a subject, the method comprising the steps of:
assaying miR-21 expression level in a biological sample from the subject by at least one of quantitative PCR, a microarray, or in situ hybridization;
comparing miR-21 expression level in the biological sample from the subject to miR-21 expression level in a control; and
determining the subject has eosinophilic esophagitis if there is an increase in miR-21 expression level in the biological sample from the subject compared to miR-21 expression level in the control.

11. The method of claim 10, further comprising the step of developing a treatment plan if the increase in miR-21 expression level in the biological sample from the subject is at least 5-fold compared to miR-21 expression level in the control.

12. The method of claim 10, wherein the sample is selected from the group consisting of an esophageal biopsy sample, serum, and mixtures thereof.

13. The method of claim 12, wherein the sample is an esophageal biopsy sample, and the assaying the miR-21 expression level in the sample is by in situ hybridization.

14. The method of claim 12, wherein the sample is serum, and the assaying the miR-21 expression level in the sample is by quantitative PCR.

15. The method of claim 10, wherein miR-21 expression level in the biological sample from the subject is greater than 30 times that of the control.

16. A method for screening for asthma in a subject, the method comprising the steps of:
assaying miR-21 expression level in a biological sample from the subject by at least one of quantitative PCR, a microarray, or in situ hybridization;
comparing miR-21 expression level in the biological sample from the subject to miR-21 expression level in a control; and
determining the subject has asthma if there is an increase in miR-21 expression level in the biological sample from the subject compared to miR-21 expression level in the control.

17. The method of claim 16, further comprising the step of developing a treatment plan if the increase in miR-21 expression level in the biological sample from the subject is at least 5-fold compared to miR-21 expression level in the control.

18. The method of claim 16, wherein the sample is serum.

19. The method of claim 16, wherein assaying miR-21 expression level in the sample is by quantitative PCR.

20. A method of treating a subject having or suspected of having a Th2-type inflammatory disease, the method comprising the step of:
administering at least one anti-inflammatory agent to the subject if miR-21 expression level in a biological sample from the subject is increased at least 5-fold relative to miR-21 expression level in a control.
